(19) United States
(12) Patent Application Publication
(10) Pub. No.: US 2012/0052592 A9
(43) Pub. Date: Mar. 1, 2012

(54) METHOD FOR DETERMINING PROGNOSIS OF ACUTE CENTRAL NERVOUS SYSTEM DISORDER

(75) Inventors: Tsuyoshi Maekawa, Yamaguchi (JP); Tomonori Izumi, Yamaguchi (JP); Yasutaka Oda, Yamaguchi (JP); Yuki Akiyoshi, Tokyo (JP)

(73) Assignees: NANO SOLUTION, INC.,, Tokyo (JP); YAMAGUCHI UNIVERSITY, Yamaguchi (JP)

(21) Appl. No.: 12/532,245

(22) PCT Filed: Mar. 27, 2008
(86) PCT No.: PCT/JP2008/055930

§ 371 Date: Nov. 29, 2010

(30) Foreign Application Priority Data
Mar. 30, 2007 (JP) 2007-091600

(51) Int. Cl. G01N 33/566; C07K 16/06; C07K 16/18

(52) U.S. Cl. 436/501; 530/389.1

(57) ABSTRACT

The present invention has its main object of providing a method for determining prognosis scientifically by searching an early marker for predicting neurological prognosis in order to grasp the disease state of a patient with an acute central nervous system disorder in the early stage and enabling an appropriate treatment to be performed. The method for determining the prognosis is provided in which the expression level of SH3BGRL3 in the biological fluid of the patient within 48 hours after resuscitation from cardiopulmonary arrest is measured, and the prognosis of the disorder classified into a good prognosis group and a poor prognosis group depending on the expression level or the presence or absence of the expression based on the Glasgow Outcome Scale (GOS) is predicted.

### TECHNICAL FIELD

The present invention relates to a method for determining prognosis of an acute central nervous system disorder, a specific antibody for measuring SH3 domain binding glutamic acid-rich protein like 3 (SH3BGRL3) which is expressed in a biological fluid such as blood or cerebrospinal fluid (CSF) of the patients with an acute central nervous system disorder, a method for determining prognosis of the acute central nervous system disorder using the antibody, and further, a kit utilized for determination.

### BACKGROUND ART

Recently, together with increase in the number of the patients with the acute central nervous system disorder and issues surrounding organ transplant, systems capable of scientifically proving the determination of prognosis of the patients with the acute central nervous system disorder have been longed for. Human death is so defined as irreversible cessation of heart (circulation), lung (breathing), and brain (central nervous system) functions that are the most important for survival. In Japan, although cardiac arrest has conventionally been placed a particular emphasis, with the development of life-support machines, human being can survive even with the heart arrest, and now, the concept of brain arrest (death) as human death has been generalized. However, the determination of brain death is also difficult and there remains many problems including such as whether profound coma, pupillary enlargement, absence of cephalic reflexes, loss of brain waves, and cessation of spontaneous respiratory should be acknowledged as overall irreversible arrest of brain functions although these are defined as the brain death.

The acute central nervous system disorder is an acute brain disorder resulting from cerebral ischemia with cardiopulmonary arrest or accompanying ischemic reperfusion after return of circulation, and the causes thereof are exemplified by cardiopulmonary arrest caused by subarachnoid hemorrhage, hypoxia, nitrogen poisoning, near-drowning, traumatic injury, and the like other than cardiogenic causes occurring to the patients with heart disease. Among the patients suffering from a severe acute central nervous system disorder, only about a few percent to 30 percent of them can return to society, which shows the importance of grasping the disease state early and providing appropriate treatment. Conventionally, the central nervous system disorder is diagnosed from abnormal findings in brain imaging tests or electrophysiological tests, decrease in cerebral blood flow or in oxygen saturation, and the like. However, these determination methods do not always reflect disease states or prognosis. Regarding disturbed consciousness, although there is a determination criterion called Glasgow Coma Scale (GCS) in which three items that are eye-opening, verbalized response, and exercise response are represented in grades and the lower grade shows more seriously disturbed consciousness, this criterion does not determine the prognosis. Further, Glasgow Outcome Scale (GOS) that determines the prognosis against the patients 6 months later is not in the acute period of the disease and therefore, the reality is that it is too late.

As a method for determining the high degree of risk or poor prognosis and for treating the patients with cardiac infarction more appropriately, a method of measuring and analyzing three types of markers, that are NT-ProBNP (N-terminal fragment of ProBNP) that is a neurohormone marker, troponin T that is an ischemic marker, and CRP (C-reactive protein) that is an inflammatory marker has been disclosed (Patent Document 1).

SH3BGRL3 was found from a human fibroblast cell as a protein that inhibits cell-lysing activity or cytostatic activity of tumor necrosis factor α (TNF α) against a tumor necrosis factor sensitive cell (Patent Document 2). This protein has been found to be the gene positioned on p 34.3 to 35 on chromosome 1 as SH3BGRL3 and has also been found to have 93 amino acids (Non-Patent Document 1).

### Patent Document 1: Japanese Patent Registration No. 3783002;

### Patent Document 2: Japanese Laid-Open Patent Application No. 6-256397;

Non-Patent Document 1: Mazzocco, M. et al. Biochem. Biophys. Res. Commun. 285: 540-545, 2001

### DISCLOSURE OF THE INVENTION

### Object to be Solved by the Invention

Conventionally, determination of the prognosis of the brain disorder of the patients with the acute central nervous system disorder is made 6 months later. However, in order to perform appropriate treatment, it is necessary to grasp the disease state in the early stage. Under such circumstances, the main object to be solved is to search an early marker for predicting the neurological prognosis of the patients with the acute central nervous system disorder and to provide methods for scientifically determining the prognosis.

### Means to Solve the Object

The present inventors have found that as a protein that inhibits the cell-lysing activity or cytostatic activity of TNF α, SH3BGRL3 found from the human fibroblast cell can be an early marker for predicting the neurological prognosis, thereby completing the present invention.

Specifically, the present invention provides (1) to (11) as mentioned below.

(1) A method for determining prognosis of an acute central nervous system disorder, the method comprising measuring the expression level of SH3BGRL3 in a biological fluid of a patient collected within 48 hours after resuscitation from cardiopulmonary arrest.

(2) The method for determining prognosis according to (1), wherein the expression level of SH3BGRL3 in a biological fluid is measured by using an antibody which specifically binds to SH3BGRL3.

(3) The method for determining prognosis according to (1) or (2), wherein the prognosis of the disorder is predicted into five categories based on Glasgow Outcome Scale (GOS) by classifying the expression level of SH3BGRL3 in a biological fluid into five categories.

(4) The method for determining prognosis according to any one of (1) to (3), wherein the biological fluid is CSF.

(5) The method for determining prognosis according to (3), wherein neurological prognosis 3 to 6 months after resuscitation from cardiopulmonary arrest is determined in the acute period within 1 week after resuscitation from cardiopulmonary arrest by defining good recovery (GR) or moderate disability (MD) in GOS as good prognosis, and defining severe disability (SD), persistent vegetative state (PVS) or death (D) as poor prognosis.

(6) An antibody used for determining prognosis of an acute central nervous system disorder according to any one of (1) to (5).

(7) The antibody according to (6),

wherein the antibody specifically binds to SH3BGRL3 and,

the antibody is obtained from serum or a tissue from the animal, immunized with a peptide having antigenicity that is selected from either of (a) peptide consisting of the amino acid sequence shown by SEQ ID No: 2 in the sequence listing; (b) peptide consisting of an amino acid sequence in which one or several amino acids are deleted, substituted, or added in the amino acid sequence shown in SEQ ID No: 2 in the sequence listing.

(8) The antibody according to (6),

wherein the antibody specifically binds to SH3BGRL3 and,

the antibody is obtained from serum or a tissue from the animal, immunized with a peptide having antigenicity that is selected from either of (a) peptide consisting of the amino acid sequence shown in SEQ ID No: 3 in the sequence listing; (b) peptide consisting of an amino acid sequence in which one or several amino acids are deleted, substituted, or added in the amino acid sequence shown in SEQ ID No: 3 in the sequence listing.

(9) The antibody according to (6), wherein the antibody obtained from the tissue from the animal according to (7) or (8) is obtained by utilizing a cell fusion technique, a genetic recombination technique, or a protein expression technique that utilize said tissue.

(10) A kit used for the method for determining prognosis according to any one of (1) to (5), wherein the kit includes SH3BGRL3 antibody and is for determining prognosis of a patient with an acute central nervous system disorder including a patient after resuscitation from cardiopulmonary arrest.

(11) A medicinal composition for treating an acute central nervous system disorder having expression-inhibiting action on SH3BGRL3 in a biological fluid.

### EFFECT OF THE INVENTION

Since the present invention enables prediction of the neurological prognosis of the patients with the acute central nervous system disorder that includes necessity for assisting the patients with the acute central nervous system disorder in their subsequent daily life and possibility for their returning to society in the early stage, to those with good prognosis obtained, possibility of their returning to society at an early date can be enhanced since appropriate treatment can be provided to them. Or the present invention can greatly contribute to the solution for the future organ transplant issues since the present invention can provide one of the scientific grounds for brain-death determination for donors that are the problems for medical transplantation.

### BEST MODE OF CARRYING OUT THE INVENTION

The present invention provides a method for determining prognosis of the acute central nervous system disorder, wherein the method for determining the prognosis is measured, comprising measuring the expression level of SH3BGRL3 in a biological fluid obtained from the patients within 48 hours after resuscitation from cardiopulmonary arrest.

The information on the gene of SH3BGRL3 is known as human SH3BGRL3 and its nucleotide sequence is registered in the gene database of National Center for Biotechnology Information of the United States (NCBI) with the accession number of NM_{—}031286 and its amino acid sequence is registered with the accession number of NP_{—}112576. The human SH3BGRL3 is the gene positioned on p 34.3 to 35 on chromosome 1 and it is a protein with low molecular weight consisting of 93 amino acids shown in the SEQ ID NO: 1 that belongs to glutaredoxin (GRX) family. And it has the same amino acid sequence as that of tumor necrosis factor α inhibitory protein (TIP-B1) (Xu, C. et al, FEBS Letters 579: 2788-2794, 2005).

The inventors of the present invention did proteomic research on biological phenomena and through the research, they have found the protein present in the biological fluid of the patients with the acute central nervous system disorder after resuscitation from cardiopulmonary arrest.

In the proteomic research, analysis of proteins in a large-scale is a major problem. In order to solve it, an instrument in which nano-flow liquid chromatography and amass spectrometer are directly linked is widely used. The specific protein related to the present invention has also been found by the separating and analyzing techniques using the instrument. It is desirable to use the nano-flow liquid chromatography in which beads with a diameter of 1 to 5 μm are filled in a tiny column (a column whose inside diameter is, for example, 100 to 200 μm and whose inside diameter at the outlet port is narrower, for example, 0.2 to 0.5 μm). For example, as a commercially available instrument, fully automatic nano-flow liquid chromatography (manufactured by Nano Solution INC.) is exemplified. Although the elution rate from the column is preferably 10 to 200 nl/min, 30 to 100 nl/min is more preferred. It is desirable to set this flow rate under the optimum conditions depending on target proteins, kinds of beads to be filled in the column, or eluates.

The eluted peptides are detected sequentially and continuously by the directly linked mass spectrometer. With the mass spectrometer, the kinds of proteins and the expression level of each protein can be compared for each sample. For example, by comparing the protein patterns in the biological fluid of the patients with the acute central nervous system disorder and those of the normal people, the proteins that can be found only in the patients or the proteins that have great difference in their expression levels can be detected.

From the protein group specifically expressed in the biological fluid of the patients with the acute central nervous system disorder, biomarkers for predicting the prognosis can be selected. For quantifying the expression level of the biomarkers in the biological fluid, the immunological method that uses antibodies which specifically bind to the corresponding biomarkers is considered to be desirable. The expression level in the biological fluid of SH3BGRL3 that is one of the biomarkers can be measured using the antibody which was raised against peptides that were synthesized based on the amino acid sequences of the antigen-determining sites of SH3BGRL3 and specifically binds to the peptides, thereby measuring the complex of the antibody and the SH3BGRL3 in the biological fluid by the immunological method. The peptide as an immunogen consists of the amino acid sequence with at least 8 amino acids and preferably, not less than 10 amino acids.

A peptide for producing the antibody has the amino acid sequence of, for example, SEQ ID NO: 2 or SEQ ID NO: 3. In the sequence, a peptide with one or several amino acid residues deleted, substituted, or added may be used. Such variants have the amino acid sequences according to SEQ ID NO: 2 or SEQ ID NO: 3 in the sequence listing and the amino acid sequence that has identity of at least not less than 70%, preferably not less than 80%, and more preferably not less than 90%.

The antibody of the present invention is the antibody that recognizes the peptide and it specifically binds to SH3BGRL3 in the biological fluid thereby capable of measuring SH3BGRL3.

The antibody of the present invention can be produced using known methods. This antibody may be any of a polyclonal antibody obtained from general antiserum, a monoclonal antibody produced by utilizing hybridoma, or an antibody obtained by utilizing gene-recombination technology or protein expression technology. The antibody obtained by utilizing the gene-recombination technology or protein expression technology can be produced by the following method. That is, it can be produced by a method comprising obtaining lymphocyte cells from immunized animals, preparing cDNA using mRNA from the lymphocytes as a template, amplifying the cDNA to insert into an appropriate vector, transferring the vector into Escherichia coli and the like to express an antibody protein. In addition, as long as the property of recognizing SH3BGRL3 is not lost, the low-molecular antibodies, the antibody fragments such as modified antibodies and the like, and the artificially produced antibodies by utilizing gene-recombination technology such as phage display and the like, and cell-free protein expression technology and the like in the test tubes may be used.

The antibody of the present invention can be obtained by administering the peptide to animals subcutaneously or intraperitoneally using an appropriate adjuvant as required to sensitize the animals and by collecting blood from the sensitized animals, then separating the serum by a known method. As animals to be immunized, although mammals or avians including rabbits, rats, mice, monkeys, sheep, chickens, and the like are exemplified, other animals may also be used and they are not particularly limited. As the antibodies, although separated serum may be used as anti-SH3BGRL serum as-is, the antibodies can also be isolated from separated serum by an affinity purification method by adsorbing the specific antibodies to the column in which the peptide including the antigen determining sites of SH3BGRL is immobilized.

Although the present invention relates to measuring the expression level of SH3BGRL3 in the biological fluid using the antibodies which specifically bind to SH3BGRL3, measurement can be performed following the known method for measuring the protein. For example, immunological measuring methods including Western blotting, dot-blotting, immunoprecipitation, EIA (enzyme-immuno assay, ELISA: enzyme-linked immunosorbent assay), RIA (radio-immuno assay), FIA (fluorescent immuno assay), immunological cell staining, and the like are exemplified.

In the above mentioned measuring methods, the antibodies which specifically bind to SH3BGRL3 or the secondary antibodies recognizing these antibodies are measured by labeling with detectable substances. As directly detectable labels, radioactive isotope, fluorescent labels such as FITC and rhodamine, and the like are exemplified and as indirectly detecting labels, enzyme labels such as horseradish peroxidase (POD), alkaline phosphatase (ALP) and the like, affinity labels such as biotin, avidin and the like, and oligonucleotide and the like can be exemplified. As indirect detection of labels, chromogenic methods utilizing the reaction of labeling enzyme, chemiluminescent methods by Enhanced Chemi Luminescence (ECL), nucleic acid amplification methods such as polymerase chain reaction, and the like are exemplified. These labeling methods and detecting methods may be used in combinations, and the labeling methods and detecting methods may be performed by already known methods.

The method of the present invention includes the step of measuring the expression level of SH3BGRL3 in the samples obtained from the patients with the acute central nervous system disorder. As the biological fluids used as samples, blood, urine, CSF, lymph, saliva, sweat, and the like are exemplified. It is desirable that the obtained samples are kept refrigerated or cryopreserved as soon as possible until use.

In the present invention, “determining prognosis” can also be rephrased as “showing prognosis of a prescribed period in the patient treatment plans after resuscitation from cardiopulmonary arrest”.

The neurological prognosis of the patients with the acute central nervous system disorder is determined based on the results of measuring the expression level of SH3BGRL3 in the samples collected from the biological fluid of the patients within 1 week, preferably within 48 hours after resuscitation from cardiopulmonary arrest. In the method of determining prognosis of the present invention, although the period for getting a final determination result of the neurological prognosis is not specifically limited as long as the determination is made based on the biological fluid of the patients within 1 week, preferably 48 hours after resuscitation from cardiopulmonary arrest, the determination is preferably made in the acute period within 1 week after resuscitation from cardiopulmonary arrest.

The prognosis determined by the method of the present invention means the state of the patients within 3 to 12 months after resuscitation from cardiopulmonary arrest, and most appropriately, it means the state of the patients 6 months thereafter. In the present invention, the state of the patients means the state of the secondary disease of the brain disorder.

The determination of the prognosis of the present invention, in addition to measuring the expression level of SH3BGRL3 of the present invention, may include the doctors' getting the determination results of the prognosis comprehensively including other clinical findings.

The evaluation for determination is made with the expression level of SH3BGRL3 in the biological fluid. It is effective to determine the prognosis by making the expression level of SH3BGRL3 correspond to the secondary disease classification of the brain disorder called GOS. GOS was advocated by Jennett, B et al. (Jennett, B., Lancet 1:480, 1975) in 1975 as a method of simply expressing the secondary disease of the brain disorder. Since many elements are involved such as physical elements, mental elements, rehabilitation, acceptance at home and in society, and the like to objectively evaluate the secondary disease of the brain disorder, it is characterized by adoption of the method for feasible evaluation, not detailed classification. It is classified into 5 categories as shown in Table 1, and according to the present invention, regarding the patients with the acute central nervous disorder, evaluation is so made that the states of GR and MD in GOS are classified as good prognosis, while the states of SD, PVS, and D in GOS are classified as poor prognosis.

In the method of the present invention, to be specific, the expression levels of SH3BGRL3 in the biological fluid of the patients are classified into 1 to 5 categories and depending on the expression levels, they can be made to correspond to each classification of GOS. In the present invention, when the expression level of SH3BGRL3 is high, it can be determined that the secondary disease of the acute central nervous system disorder is severe and the prognosis is poor. It is preferable in the determination of the present invention that in measuring the expression level of SH3BGRL3, when SH3BGRL3 is detected, it can be determined as poor prognosis, while when SH3BGRL3 is not detected, it can be determined as good prognosis.

TABLE 1

| Classification | Definition |
|---|---|
| Death (D) | Except death by other organ |
| | disorders in the acute period. |
| Persistent vegetative | Eye-opening, persistent |
| state (PVS) | sleep-wake cycle, absence of |
| | function in the cerebral cortex. |
| Severe disability (SD) | Retained consciousness but |
| | dependent daily lives on others. |
| Moderate disability (MD) | Non-dependent daily lives on |
| | others. Some hemiplegia, ataxia, |
| | intellectual disorders, memory |
| | disorders, and altered |
| | personality are allowed. |
| Good recovery (GR) | Minor neurological and |
| | psychological deficits are |
| | allowed. |

When the subjects are human beings, in general, diseases are diagnosed by doctors (also including those instructed by doctors, bd.) and the data on the expression level of SH3BGRL3 obtained by the inspection method of the present invention are helpful to the doctors for their diagnosis. Thus, the inspection method of the present invention can be also expressed as the method of collecting and providing the data helpful for the diagnosis by the doctors.

The present invention relates to detection reagents for determining the prognosis of the acute central nervous system disorder including the reagents for measuring the expression level of SH3BGRL3. Such reagents can include the ones used for the step of measuring the SH3BGRL3 level as mentioned above. For example, antibodies, staining solutions, and the like required for measuring the SH3BGRL3 level can be exemplified.

Further, making into a kit by combining the reagent for measuring the expression level of SH3BGRL3 that is the base of the determination method of the present invention and other elements are also available. In the kit, other than specific antibodies, immobilized carriers, labeled substances, substrate compounds used for detecting the labels, others including distilled water, salt, buffer solutions, protein stabilizers, preservatives, and the like can be included. Further, written instructions for explaining measuring operations can be attached to the kit.

The inventors of the present invention have revealed that the proteins such as SH3BGRL3 and the like are expressed specifically in the biological fluid of the patients with the acute central nervous system disorder of the poor prognosis. From these findings, as substances that inhibit the expression of the specific proteins, they can also be the targets for treatment and drug discovery.

For information, all the prior art documents cited in the present specification are incorporated therein as references.

### BRIEF DESCRIPTION OF DRAWINGS

FIG. 1 shows the mass spectrum of a peptide derived from SH3BGRL3 protein.

FIG. 2 shows the amino acid sequence of SH3BGRL3 protein deduced from cDNA sequence and two antigenic sites. In addition, they area human sequence (NP_{—}112576, NM_{—}031286, SEQ ID NO: 1), a mouse sequence (NP_{—}542126, NM_{—}080559, SEQ ID NO: 4) and a rat sequence (NP_{—}001100158, NM_{—}001106688, SEQ ID NO: 5).

FIG. 3 shows the graph of the antibody titer of rabbit-derived antiserum after immunization.

FIG. 4 shows the graph of the antibody titer of anti-SH3BGRL3 IgG after affinity purification.

FIG. 5 shows the picture substituted for the drawing, showing the Western blotting of CSF samples of the patients with anti-SH3BGRL3 antiserum used. It shows that the SH3BGRL3 protein with a molecular weight of 24 kDa (arrow) is specifically detected only in the patients with poor prognosis.

### EXAMPLE 1

Examples are described in the following for explaining the present invention further in detail, while the present invention is not limited to these examples.

(Protein Analysis of a Specimen of Patients with the Acute Central Nervous System Disorder)

Reagent: As a CSF specimen of the patient with cardiopulmonary resuscitation, the one collected within 48 hours after resuscitation was used. As a control specimen of CSF, the one collected at the time of lumbar anesthesia of a neurologically normal person was used.

Preparation of Reagent: 4 times as much of cold acetone was added to CSF and the precipitated protein was collected by centrifugal separation. After drying under reduced pressure, the precipitate was dissolved in a 0.1 M tris-HCl buffer solution containing 8 M urea, followed by reducing with 2 mM dithiothreitol for 1 hour at room temperature, further followed by alkylating with 5 mM iodoacetamide for 1 hour at room temperature. Reaction solution was diluted by 4 times with water and was digested with swine-derived trypsin (enzyme-substrate ratio 1:50) for 16 hours at a temperature of 37° C.

Nano-Flow Liquid Chromatography-Mass Spectrometry: Separation and accurate mass measurement of peptides in an enzyme digest was performed by a protein analysis system consisting of fully automatic nano-flow liquid chromatography (manufactured by Nano Solution INC.) and a Q-tof mass spectrometer (manufactured by Micromass Limited.). Separation of the peptides was performed, using a reversed phase column with octadodecylsilica beads (Mightysil-C18, manufactured by KANTO CHEMICAL CO., INC.) filled in a fused silica capillary with an inside diameter of 150 μm and a length of 50 mm, by increasing the acetonitrile concentration from 0% to 80% with an eluting solution (flow rate 100 nl/min) including 0.1% of formic acid after loading samples. The eluted peptides were introduced to the Q-tof mass spectrometer online, and the accurate mass of parent ion and internal fragment ion was measured.

Searching the Sequence Database and Identifying the Proteins: The obtained mass data were searched against the amino acid sequence database (RefSeq human, NCBI of US) using a sequence searching software MASCOT (manufactured by Matrix Science Ltd.) by making only searched results in which peptide sequence is determined with not less than 95% reliability belong to protein thereby making identification lists. The lists were compared among the samples of the patients with control, good prognosis, and poor prognosis, classifying the identified proteins into three that are the protein group identified in common among the three (group I), the protein group identified in common among any of the two (group II), and the protein group identified specifically to each one (group III) and the proteins identified specifically to the patients with poor prognosis in the group III were defined to be marker candidate proteins of the central nervous system disorder.

### (Result 1)

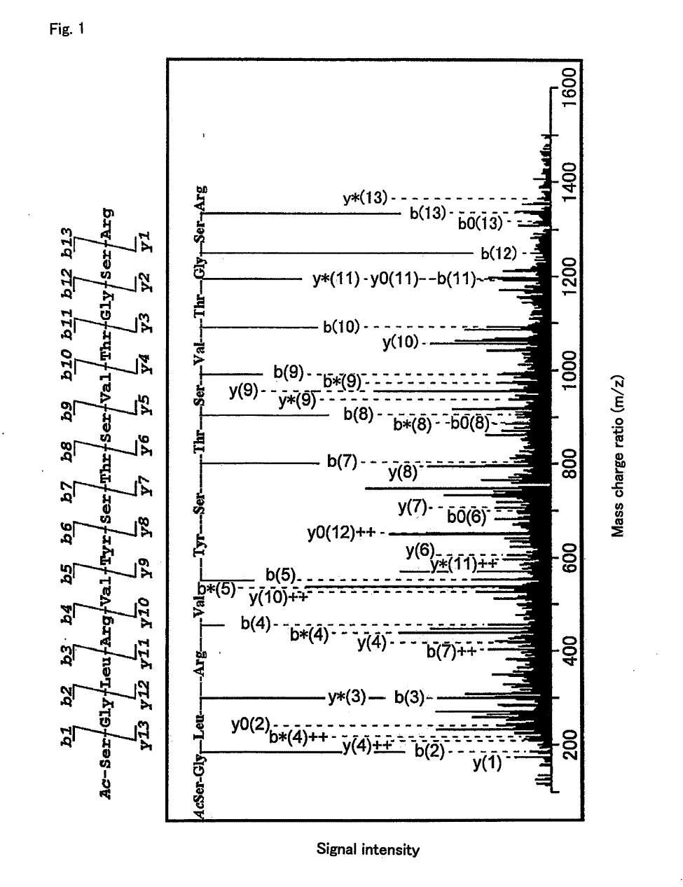

From the protein analysis of the samples of the CSF utilizing the mass spectrometry analysis, as one of the proteins specific to poor prognosis, the SH3BGRL3 protein having the amino acid sequence shown in the SEQ ID NO: 1 was identified (FIG. 1).

### EXAMPLE 2

### Production of SH3BGRL3 Antiserum

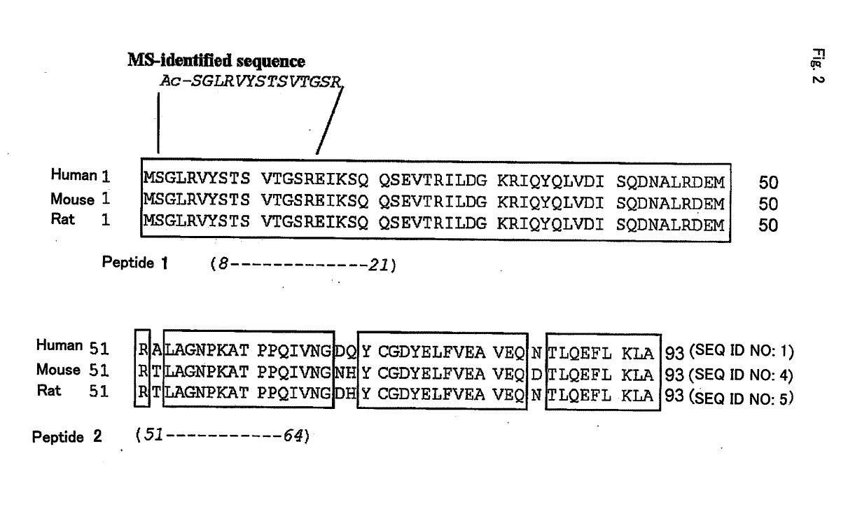

Production of Antigen: The following two kinds of peptides including partial sequences (Arg₅₁-Ile₆₄: SEQ ID NO: 2 and Ser₈-Gln₂₁: SEQ ID NO: 3) of human SH3BGRL3 protein (a full-length 93 amino-acid residues: SEQ ID NO: 1) expected from the nucleotide sequence (Mazzocco, M. et al. Biochem. Biophys. Res. Commun. 285:540-545, 2001: Non-Patent Document 1) were synthesized and after covalently coupling to carrier protein KLH (Keyhole limpet hemocyanin) through a linker, they were mixed to produce immunogen (FIG. 2).

Peptide 1:
(Cys)-Ser₈-Thr-Ser-Val-Thr-Gly-Ser-Arg-Glu-Ile-
Lys-Ser-Gln-Gln₂₁
Peptide 2:
Arg₅₁-Ala-Leu-Ala-Gly-Asn-Pro-Lys-Ala-Thr-Pro-
Pro-Gln-Ile₆₄-(Cys)

The above mentioned (Cys) represents cysteine for carrier conjugation.

Immunization Method: 400 μl of KLH bound peptide (equivalent to 200 μg of each peptide, 400 μg in total/time per rabbit) and 400 μl of adjuvant (first time; Freund complete adjuvant, second time and thereafter; Freund incomplete adjuvant) were mixed to make an emulsion, which was intracutaneously immunized to 20 to 30 portions of the back region of rabbits (Japanese White) of 14 to 20 weeks old (in the vicinity of 3 kg, 2 females). Regarding the immunization schedule, blood was collected as an examination before immunization in the first week followed by the first immunization, by the second immunization carried out in the third week, by the third immunization carried out in the fourth week, by the fourth immunization carried out in the sixth week, by the fifth immunization carried out in the seventh week, by blood collection carried out as an examination in the eighth week, and by the sixth immunization carried out after confirming titer. In the tenth week, whole blood was collected in culture tubes with serum separating agents therein, which was subjected to centrifugal separation, thereby obtaining serum.

Confirmation of Antiserum Titer: The confirmation was made by ELISA method (conducted at the time of collecting blood as an examination after five times of immunization, and at the time of collecting whole blood). Peptide was diluted to 1 μg/ml (0.5 μg/ml each) with phosphate-buffered saline (PBS), followed by dispensing it at 100 μl/well into a plate for sensitization and keeping it still overnight at a temperature of 4° C. After sensitization, the antigen solution was removed and after washing once with PBS, a blocking solution (manufactured by Medical and Biological Laboratories Co., Ltd., or MBL Co., Ltd.) was dispensed at 200 μl/well and was kept still overnight at a temperature of 4° C. A dilution series of rabbit serum before immunization and rabbit antiserum after immunization were prepared to be 100 times, 500 times, 2500 times, 12500 times, 62500 times, and blank, and the one diluted with PBS was added thereto at 100 μl/well, and reacted for 60 minutes at a temperature of 25° C. After washing, an anti-rabbit IgG-POD conjugate (product of MBL Co., Ltd.) was diluted 8000 times with a dilution buffer solution (manufactured by MBL Co., Ltd.), which was added at 100 μl/well, and reacted for 60 minutes at a temperature of 25° C. After washing, a color developing solution (manufactured by MBL Co., Ltd.) was added at 100 μl/well, developing color for 3 to 10 minutes, followed by adding 2 N sulfuric acid at 100 μl/well, thereby terminating the reaction. After termination of the reaction, absorbance was measured at the measuring wavelength of 450 nm and at the reference wavelength of 620 nm.

### (Result 2)

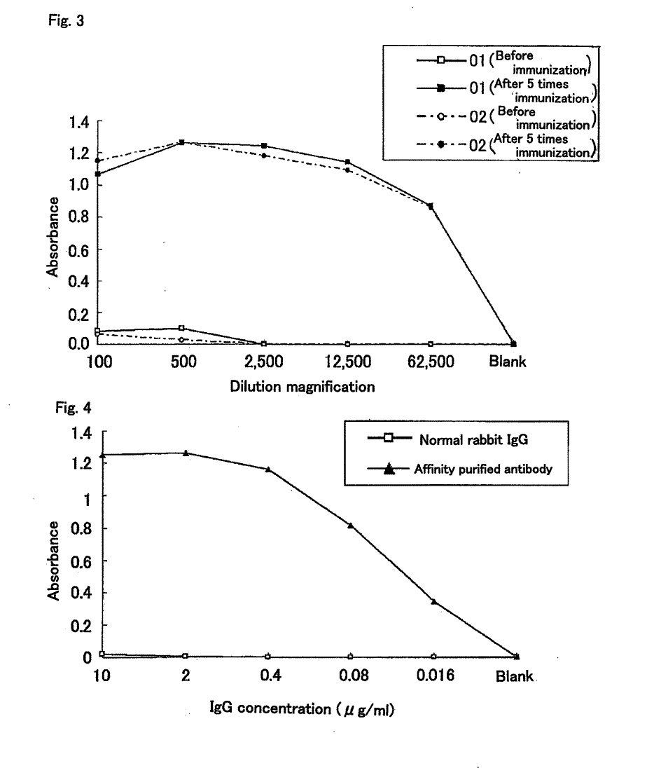

The graph of the antibody titer of the two rabbit-derived antisera was shown in FIG. 3, from which it is observed to be the antibody with sufficiently higher antibody titers compared with the rabbit sera before immunization.

### EXAMPLE 3

### Production of Anti-SH3BGRL3 Specific Antibody

Production of Antigen Peptide-Immobilized Column:

SulfoLink Kit (manufactured by Pierce Biotechnology Inc.) was used. 2 ml of gel was filled in a column and was washed with 8 ml of a coupling buffer solution. After each of 1 mg antigen peptide and 2 mg in total was dissolved in 2 ml of the coupling buffer solution, the antigen peptide was loaded onto the column and was stirred with a rotator for 15 minutes at room temperature. After further kept still for 30 minutes at room temperature, the column was washed with 8 ml of the coupling buffer solution. Next, 2 ml of cysteine (7.9 mg/ml) was loaded onto the column and was stirred with a rotator for 15 minutes at room temperature. After further kept still for 30 minutes at room temperature, the column was washed with 6 ml of the buffer solution for washing and after substituting the buffer solution for washing with a buffer solution for affinity adsorption, it was preserved at a temperature of 4° C. Immobilization reaction efficiency was verified by quantifying SH group in a peptide solution before and after reaction with Ellman reagent.

Purification of Specific Antibody: The column was equilibrated with PBS and 10 ml of the antiserum of rabbit No. 02 was loaded. After washing the column with PBS, the antibody was eluted with 0.1 M glycine-HCl (pH 2.3) whose volume is four times as much as the column volume. The eluted antibody was quickly neutralized with 1 M tris-HCl (pH 8.0) on-ice. A fraction that includes the antibody was dialyzed against 50% glycerol/PBS to obtain the anti-SH3BGRL3 specific antibody.

Confirmation of Titer of Purified Specific Antibody:

The confirmation was made by ELISA method. That is, peptide was diluted to 1 μg/ml (0.5 μg/ml each) with PBS, followed by dispensing it at 100 μl/well into a plate for sensitization and keeping it still overnight at a temperature of 4° C. After sensitization, an antigen solution was removed and after washing once with PBS, Blocking Buffer (manufactured by MBL Co., Ltd.) was dispensed at 200 μl/well and was kept still overnight at a temperature of 4° C. A dilution series of normal rabbit IgG and purified rabbit IgG were set to be 10 μg/ml, 2 μg/ml, 0.4 μg/ml, 0.08 μg/ml, 0.016 μg/ml, and blank and they were diluted with PBS, which was added at 100 μl/well, thereby reacting for 60 minutes at a temperature of 25° C. After washing, an anti-rabbit IgG-POD conjugate (product of MBL Co., Ltd.) was diluted 8000 times with the dilution buffer (manufactured by MBL Co., Ltd.), which was added at 100 μl/well, and reacted for 60 minutes at a temperature of 25° C. After washing, the color developing solution (manufactured by MBL Co., Ltd.) was added at 100 μl/well, developing color for 3 to 10 minutes, followed by adding 2N sulfuric acid at 100 μl/well, thereby terminating the reaction. After termination of the reaction, absorbance was measured at the measuring wavelength of 450 nm and at the reference wavelength of 620 nm.

### (Result 3)

It is clearly observed from the graph of FIG. 4 that the IgG antibody having the higher antibody titer was obtained by affinity purification.

### EXAMPLE 4

### Western Blotting of CSF Samples of Patients

Western Blotting: Samples of CSF was mixed with an equivalent amount of a solution for sample preparation (4% SDS, 10% 2-mercaptoethanol, 20% glycerol, 0.2% bromophenol blue) and heat-treated for 3 minutes at a temperature of 100° C. For separation, 10% polyacrylamide gel was used, and after loading 2 to 5 μl of the sample per lane, electrophoresis was performed at 20 mA for 90 minutes in 25 mM tris-192 mM glycine buffer solution (pH 8.3) that includes 0.1% SDS. The gel after the electrophoresis was transferred to a blotting apparatus and was subjected to electroblotting at 50 mA for 90 minutes onto a polyvinylidenedifluororide (PVDF) membrane that was equilibrated by 25 mM tris-192 mM glycine buffer solution (pH 8.3) that includes 20% methanol.

Antibody Staining:

The PVDF membrane after electroblotting was subjected to blocking overnight at a temperature of 4° C. in a buffer solution (TBS-T: 10 mM tris-HCl (pH 7.4), 150 mM NaCl, 0.1% Tween 20) that includes 5% skim milk. After washing the membrane with the TBS-T, anti-SH3BGRL3 antiserum diluted 1/500 or a specific antibody was added, followed by incubating for 1 hour at room temperature, by further washing with the TBS-T, followed by adding peroxidase-conjugated anti-rabbit IgG antibody diluted 1/2000 thereby incubating for 1 hour at room temperature. SH3BGRL3 protein was visualized by adding a chemiluminescent reagent (ECL Plus, GE Health Science Co., Ltd.) to the membrane and by exposing it to an X-ray film.

### (Result 4)

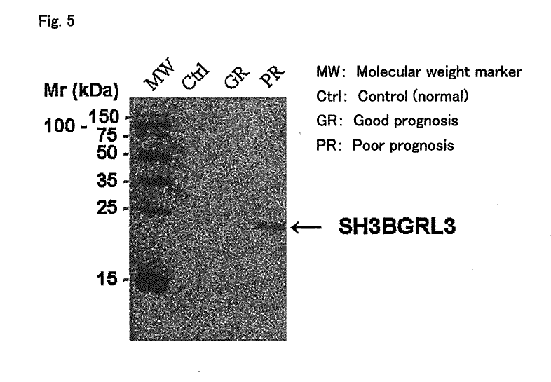

In the Western blotting analysis of the sample of the CSF of the patients using the anti SH3BGRL3 antiserum, the SH3BGRL3 protein whose molecular weight is 24 kDa (that includes non-specified posttranslation modification) was specifically detected only in the sample of the patients with poor prognosis (FIG. 5).

Relationship between the expression of SH3BGRL3 and the symptom of the patients is as follows.

CSF of normal control patients: Western blot positive 0 case out of 9 cases

CSF of patients with good prognosis group: Western blot positive 1 case out of 5 cases

CSF of patients with poor prognosis group: Western blot positive 6 cases out of 7 cases

### INDUSTRIAL APPLICABILITY

Since the present invention enables prediction of neurological prognosis of the patients with the acute central nervous system disorder in the early stage, by providing appropriate treatment to those with determination of good prognosis obtained, possibility of their returning to society can be enhanced. Or to those with determination of poor prognosis obtained, since the determination can be the one scientific ground for brain-death diagnosis for donors that is the problem for medical transplantation, cooperation for donating organ transplant can be requested promptly.

1. A method for determining prognosis of an acute central nervous system disorder, the method comprising measuring the expression level of SH3BGRL3 in a biological fluid of a patient collected within 48 hours after resuscitation from cardiopulmonary arrest.

2. The method for determining prognosis according to claim 1, wherein the expression level of SH3BGRL3 in a biological fluid is measured by using an antibody which specifically binds to SH3BGRL3.

3. The method for determining prognosis according to claim 1 or 2, wherein the prognosis of the disorder is predicted into five categories based on Glasgow Outcome Scale (GOS) by classifying the expression level of SH3BGRL3 in a biological fluid into five categories.

4. The method for determining prognosis according to any one of claims 1 to 3, wherein the biological fluid is cerebrospinal fluid.

5. The method for determining prognosis according to claim 3, wherein neurological prognosis 3 to 6 months after resuscitation from cardiopulmonary arrest is determined in the acute period within 1 week after resuscitation from cardiopulmonary arrest by defining good recovery (GR) or moderate disability (MD) in GOS as good prognosis, and defining severe disability (SD), persistent vegetative state (PVS) or death (D) as poor prognosis.

6. An antibody used for determining prognosis of an acute central nervous system disorder according to any one of claims 1 to 5.

7. The antibody according to claim 6,
wherein the antibody specifically binds to SH3BGRL3 and, the antibody is obtained from serum or a tissue from the animal, immunized with a peptide having antigenicity that is selected from either of (a) peptide consisting of the amino acid sequence shown by SEQ ID No: 2 in the sequence listing; (b) peptide consisting of an amino acid sequence in which one or several amino acids are deleted, substituted, or added in the amino acid sequence shown in SEQ ID No: 2 in the sequence listing.

8. The antibody according to claim 6,
wherein the antibody specifically binds to SH3BGRL3 and, the antibody is obtained from serum or a tissue from the animal, immunized with a peptide having antigenicity that is selected from either of (a) peptide consisting of the amino acid sequence shown in SEQ ID No: 3 in the sequence listing; (b) peptide consisting of an amino acid sequence in which one or several amino acids are deleted, substituted, or added in the amino acid sequence shown in SEQ ID No: 3 in the sequence listing.

9. The antibody according to claim 6, wherein the antibody obtained from the tissue from the animal according to claim 7 or 8 is obtained by utilizing a cell fusion technique, a genetic recombination technique, or a protein expression technique that utilize said tissue.

10. A kit used for the method for determining prognosis according to any one of claims 1 to 5, wherein the kit includes SH3BGRL3 antibody and is for determining prognosis of a patient with an acute central nervous system disorder including a patient after resuscitation from cardiopulmonary arrest.

11. A medicinal composition for treating an acute central nervous system disorder having expression-inhibiting action on SH3BGRL3 in a biological fluid.


SEQUENCE LISTING (5 sequences)

SEQ ID NO: 1 - protein, 93 aa, Homo sapiens
  PEPTIDE (1)..(93)
MSGLRVYSTSVTGSREIKSQQSEVTRILDGKRIQYQLVDISQDNALRDEMRALAGNPKATPPQIVNGDQYCGDYELFVEAVEQNTLQEFLKLA

SEQ ID NO: 2 - protein, 14 aa, Homo sapiens
  DOMAIN (1)..(14)
RALAGNPKATPPQI

SEQ ID NO: 3 - protein, 14 aa, Homo sapiens
  DOMAIN (1)..(14)
STSVTGSREIKSQQ

SEQ ID NO: 4 - protein, 93 aa, Mus musculus
MSGLRVYSTSVTGSREIKSQQSEVTRILDGKRIQYQLVDISQDNALRDEMRTLAGNPKATPPQIVNGNHYCGDYELFVEAVEQDTLQEFLKLA

SEQ ID NO: 5 - protein, 93 aa, Rattus norvegicus
MSGLRVYSTSVTGSREIKSQQSEVTRILDGKRIQYQLVDISQDNALRDEMRTLAGNPKATPPQIVNGDHYCGDYELFVEAVEQNTLQEFLKLA